(19) United States
(12) Patent Application Publication
(10) Pub. No.: US 2024/0108878 A1
(43) Pub. Date: Apr. 4, 2024

(54) CONTROLLING ELECTRODE POTENTIALS

(71) Applicant: Medtronic, Inc., Minneapolis, MN (US)

(72) Inventors: Kristin N. Hageman, Dayton, MN (US); Aleksandra P. Kharam, Maple Grove, MN (US); David A. Dinsmoor, North Oaks, MN (US); Shawn C. Kelley, Shoreview, MN (US)

(21) Appl. No.: 18/478,439

(22) Filed: Sep. 29, 2023

Related U.S. Application Data
(60) Provisional application No. 63/377,836, filed on Sep. 30, 2022.

(51) Int. Cl. A61N 1/02; A61N 1/36

(52) U.S. Cl. CPC A61N 1/025; A61N 1/36135; A61N 1/36175; A61N 1/36185; A61N 1/37235

(57) ABSTRACT

Example techniques, devices, and systems are described herein. An example device includes stimulation generation circuitry, sensing circuitry, and processing circuitry. The processing circuitry is configured to control the stimulation generation circuitry to generate a first stimulation signal having a first stimulation recharge parameter for delivery to target anatomy and receive from the sensing circuitry a sensed evoked response signal. The processing circuitry is configured to analyze the sensed evoked response signal for one or more artifacts and adjust, based on the one or more artifacts, the first stimulation recharge parameter to determine a second stimulation recharge parameter. The processing circuitry is also configured to control the stimulation generation circuitry to generate a second stimulation signal having the second stimulation recharge parameter for delivery to the target anatomy.

This application claims the benefit of U.S. Provisional Patent Application No. 63/377,836, filed Sep. 30, 2022, the entire content of which is incorporated herein by reference.

### TECHNICAL FIELD

This disclosure generally relates to medical devices, and more specifically, to devices configured to sense bioelectrical signals.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the spinal cord, the brain, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

Electrical stimulation may be delivered to a patient by the medical device in a train of electrical pulses, and parameters of the electrical pulses may include a frequency, an amplitude, a pulse width, and a pulse shape. An evoked compound action potential (ECAP) is a synchronous firing of a population of neurons which occurs in response to the application of a stimulus including, in some cases, an electrical stimulus by a medical device. The ECAP may be detectable as being a separate event from the stimulus itself, and the ECAP may reveal characteristics of the effect of the stimulus on the nerve fibers.

Evoked response style sensing (e.g., electrically evoked compound action potential (EECAP), evoked resonant neural activity (ERNA), electromyogram (EMG), evoked compound muscle action potential (eCMAP), or the like) has proven to be useful in assessing dosing response for neuromodulation therapies as well as potentially giving insights into a disease state of a patient. This sensed data may serve as control signals for closed loop stimulation therapies.

### SUMMARY

A medical device, such as an implantable medical device (IMD) may provide stimulation therapy to target anatomy of a patient by delivering a stimulation signal via electrodes. Such a stimulation signal may be configured to include an active recharge phase, a passive recharge phase, or a hybrid active/passive recharge phase. In some examples, the recharge phases may be configured to relatively charge balance the stimulation signals. In such examples, the stimulation signal may maintain net charge neutrality, and prevent charge build up on direct current (DC) blocking capacitors of the IMD (if such DC blocking capacitors are used) as well as charge build up on an electrode-tissue (or anatomy) interface. In some examples, the recharge phases may be configured to intentionally not charge balance the stimulation signal.

An active recharge phase may include a current of an opposite polarity after a stimulation pulse that may be applied across the stimulation electrodes. A passive recharge phase may be configured to allow built-up charge to passively dissipate by the IMD connecting an electrode interface via the DC blocking capacitors to a low impedance, such as ground.

Stimulation signals may cause an evoked response in the target anatomy. This evoked response may be sensed by sensing circuitry of the IMD and may be used by the IMD to provide closed loop stimulation. In some examples, the sensed evoked response signal may include one or more artifacts which may be caused by stimulation pulses. It may be desirable to reduce the amplitude, duration, area under the curve, or the like of such artifacts to facilitate the IMD properly analyzing the actual evoked response, for example, when the IMD may change stimulation parameters based on the sensed evoked response signal, in order to maintain proper therapeutic stimulation.

In general, systems, devices, and techniques are described for controlling electrode operating potential by monitoring and analyzing characteristics of an artifact in a sensed evoked response signal and adjusting stimulation recharge parameters in a manner that minimizes or reduces the artifact and/or optimizes or improves the electrode potential within desired limits of operation. Such techniques may be particularly useful because a DC connection on the electrodes is not necessary to assess the potentials of the electrodes. Such information may be obtained (or a surrogate therefore may be obtained) from artifact parameters (for example, a slope of the artifact) which may be sensed by a sense amplifier of the IMD. These techniques may also be useful, as a medical device may optimize or improve stimulation recharge parameters such that an operating point of an electrode after stimulus is closer to the electrode's open circuit potential (OCP) or stable point of operation.

An example device includes stimulation generation circuitry configured to generate a first stimulation signal, according to a set of parameters to be delivered to target anatomy of a patient via a stimulation electrode configuration from a plurality of electrodes, the set of parameters comprising a first stimulation recharge parameter; sensing circuitry configured to sense an evoked response signal responsive to the stimulation signal; processing circuitry communicatively coupled to the stimulation generation circuitry and the sensing circuitry, the processing circuitry being configured to: control the stimulation generation circuitry to generate the first stimulation signal having the first stimulation recharge parameter for delivery to the target anatomy; receive from the sensing circuitry the sensed evoked response signal; analyze the sensed evoked response signal for one or more artifacts; adjust, based on the one or more artifacts, the first stimulation recharge parameter to determine a second stimulation recharge parameter; and control the stimulation generation circuitry to generate a second stimulation signal having the second stimulation recharge parameter for delivery to the target anatomy.

An example method includes controlling, by processing circuitry, stimulation generation circuitry to generate the first stimulation signal having a first stimulation recharge parameter for delivery to target anatomy of a patient; receiving, by the processing circuitry and from sensing circuitry, a sensed evoked response signal; analyzing, by the processing circuitry, the sensed evoked response signal for one or more artifacts; adjusting, by the processing circuitry and based on the one or more artifacts, the first stimulation recharge parameter to determine a second stimulation recharge parameter; and controlling, by the processing circuitry, the stimulation generation circuitry to generate a second stimulation signal having the second stimulation recharge parameter for delivery to the target anatomy.

An example non-transitory computer readable medium includes instructions, which, when executed, cause processing circuitry to: control stimulation generation circuitry to generate a first stimulation signal having a first stimulation recharge parameter for delivery to target anatomy; receive from sensing circuitry a sensed evoked response signal; analyze the sensed evoked response signal for one or more artifacts; adjust, based on the one or more artifacts, the first stimulation recharge parameter to determine a second stimulation recharge parameter; and control the stimulation generation circuitry to generate a second stimulation signal having the second stimulation recharge parameter for delivery to the target anatomy.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example system that includes an implantable medical device (IMD) configured to deliver spinal cord stimulation (SCS) therapy and an external programmer, in accordance with one or more techniques of this disclosure.

FIG. 2A is a block diagram illustrating an example combination of components of an IMD, in accordance with one or more techniques of this disclosure.

FIG. 2B is a block diagram illustrating an example combination of components of an example external programmer, in accordance with one or more techniques of this disclosure.

FIGS. 3A-3B are conceptual diagrams illustrating an example passive charge balancing waveform and an example active charge balancing waveform, respectively.

FIG. 4 is a conceptual diagram illustrating an example stimulus waveform having passive recharge balancing.

FIG. 5 is a conceptual diagram illustrating examples of stimulation artifacts superimposed on top of an example tissue neural response signal.

FIG. 6 is a conceptual diagram illustrating example simplified double layer capacitor models for an electrode-tissue interface.

FIG. 7 is a schematic diagram illustrating an example simplified model of implantable neurostimulation device and tissue interface during stimulation.

FIG. 8 is a schematic diagram illustrating an example device and electrode charging during a leading phase of a stimulus pulse.

FIG. 9 is a schematic diagram illustrating example device and electrode capacitors during a passive recharge phase of a stimulation signal.

FIG. 10 is a schematic diagram illustrating an example 0 volt crossing on a double layer capacitor during passive recharging.

FIG. 11 is a schematic diagram illustrating an example re-polarization of a double layer capacitor.

FIG. 12 is a conceptual diagram illustrating example artifact and electrode voltage change depending on when passive recharge stops.

FIG. 13 is a schematic diagram illustrating device and electrode capacitors discharging in an active recharge phase.

FIG. 14 is a conceptual diagram illustrating 0 volt crossing on a double layer capacitor during active recharging.

FIG. 15 is a flow diagram illustrating techniques to control artifact and electrode potential via a recharge phase.

### DETAILED DESCRIPTION

This disclosure describes examples of medical devices, systems, and techniques for controlling electrode operating potential by analyzing characteristics of an artifact in a sensed evoked response signal and adjusting stimulation recharge parameters in a manner that minimizes or reduces the artifact and optimizes or improves the electrode potential within desired limits of operation. The sensed evoked response signal may be an evoked compound action potential (ECAP), an EECAP, local field potential (LFP), ERNA, EMG, eCMAP, or any other signal that may be indicative of a physiological reaction to an electrical stimulation signal. By analyzing the sensed evoked response signal for one or more artifacts, an IMD may determine whether to and/or how to adjust stimulation recharge parameters to minimize or reduce the effects of artifacts on the sensed evoked response signal and/or to optimize or improve electrode potential.

Electrical stimulation therapy is typically delivered to a target tissue (e.g., nerves of the spinal cord, muscle, organ, or other tissue) of a patient via two or more electrodes. Parameters of the electrical stimulation therapy (e.g., electrode combination, polarity of the electrodes, voltage or current amplitude, pulse width, pulse frequency, pulse shape, number of interleaved pulses, passive recharge parameters, active recharge parameters, etc.) are selected by a clinician and/or the patient to provide relief from various symptoms, such as pain, nervous system disorders, muscle disorders, etc. Various thresholds, such as a perception threshold and/or discomfort threshold may be determined for the patient and used to select and/or recommend various stimulation parameters of the stimulation therapy.

Evoked responses are a measure of neural recruitment because each evoked response signal represents the superposition of electrical potentials generated from a population of excitable tissue (such as nerve axons) firing in response to an electrical stimulus (e.g., a stimulation pulse). Changes in a characteristic (e.g., an amplitude of a portion of the signal, such as a peak-to-peak amplitude or area under the curve of the signal) of an evoked response signal occur as a function of how many axons have been activated by the delivered stimulation pulse. For a given set of stimulation parameter values that define the stimulation signal and a given distance between the electrodes and target nerve, the detected evoked response signal may have a certain characteristic value (e.g., peak-to-peak amplitude).

In some examples, effective stimulation therapy may rely on a certain level of neural recruitment at a target nerve. This effective stimulation therapy may provide relief from one or more conditions (e.g., patient perceived pain) without an unacceptable level of side effects (e.g., overwhelming perception of stimulation).

Although the system may adjust one or more stimulation parameters according to the one or more characteristics of the sensed evoked response signal, for example, to compensate for the change in distance between electrodes and nerves, the precision of such adjustments is dependent on accurately determining the actual evoked response. For example, noise such as stimulation artifacts and/or linear or exponential background noise may interfere with accurate determinations of the magnitude of one or more peaks within the evoked response signal. Stimulation artifacts typically have amplitudes many times that of the evoked response signal and can at least partially overlap with the evoked response from nerves. Inaccurate evoked response characterization can reduce the effectiveness of using evoked response characteristic values for automatically adjusting stimulation parameters and result in less efficacious therapy for the patient. Moreover, manually identifying patient thresholds, such as a perception threshold, can be time consuming and rely on subjective feedback from the patient. Therefore, clinicians may be pressed for time when setting up stimulation, perception thresholds may be inaccurate, and patients may need to return to a clinic in order to update the stimulator programming, for example. These issues may reduce the likelihood that the patient receives efficacious therapy.

In general, it is desirable to improve biopotential sensing by reducing the impact of artifacts and improving the electrode potential such that the electrode potential is within desired limits of operation. Such improved biopotential sensing may reduce the amplitude of therapy signals, thereby resulting in lower power consumption, longer recharge intervals, and/or a longer battery life. As described herein, systems, devices, and techniques are described for controlling electrode operating potential by monitoring and analyzing characteristics of an artifact in a sensed evoked response signal and adjusting stimulation recharge parameters in a manner that minimizes or reduces the artifact and optimizes or improves the electrode potential within desired limits of operation. For example, an IMD may monitor an evoked response signal for one or more artifacts and adjust a recharging parameter of a stimulation signal based on characteristics of the one or more artifacts. As used herein, “recharging” refers to the reversal of current flow—the second phase of a biphasic stimulation signal—through stimulating electrodes to both prevent or limit tissue damage at the electrode-tissue interface, and reverse polarization developed across the electrode blocking capacitors routinely used with IMDs.

Stimulation recharge parameters may be active, passive or a combination of active and passive (e.g., hybrid active/passive). Passive recharging may significantly reduce the current draw for an IMD, thereby lengthening battery recharging intervals and/or battery life. Thus, there are benefits for utilizing passive recharge. On the other hand, passive recharge can impose timing constraints on the stimulus delivery rate as it may take a significantly longer time to remove the charge using passive recharge compared to active recharge. Further, passive recharge may result in reduced capability to sense relevant biopotentials during the long-tailed decay of the passive recharge phase.

In some examples, the evoked responses detected by an IMD may be evoked responses elicited by stimulation pulses intended to contribute to therapy of a patient or separate pulses (e.g., control pulses) configured to elicit evoked responses that are detectable by the IMD. Nerve impulses detectable as the evoked response signal travel quickly along the nerve fiber after the delivered stimulation pulse first depolarizes the nerve. If the stimulation pulse delivered by first electrodes has a pulse width that is too long, electrodes configured to sense the evoked response may sense the stimulation pulse itself as an artifact (e.g., detection of delivered charge itself as opposed to detection of a physiological response to the delivered stimulus) that obscures the lower amplitude evoked response signal.

Moreover, an IMD and/or lead(s) carrying electrodes used for stimulation and sensing may migrate within the patient, or the patient may change postures placing more or less pressure on areas containing the IMD and/or lead(s) thereby causing the IMD and/or lead(s) to be closer or farther from the target tissue than at other times. Additionally, a patient disease state may change over time. Therefore, it may be desirable to adjust stimulation recharge parameters to more accurately identify the evoked response and thereby configure the electrical stimulation in a closed loop manner to deliver more efficacious therapy.

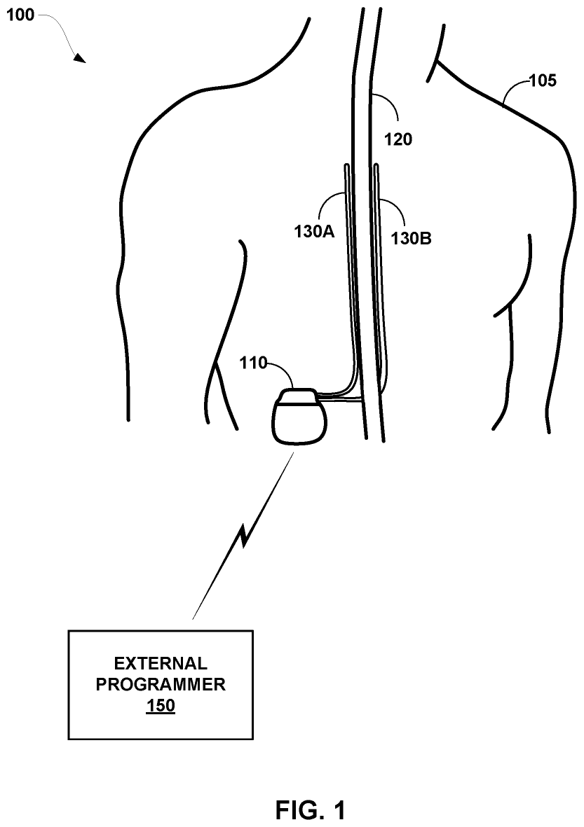

FIG. 1 is a conceptual diagram illustrating an example system 100 that includes an implantable medical device (IMD) 110 configured to deliver spinal cord stimulation (SCS) therapy and an external programmer 150, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration only, but without limitation as to other types of medical devices or other therapeutic applications of medical devices, for example, nerve stimulation, pelvic stimulation, deep brain stimulation, cardiac stimulation, or the like.

As shown in FIG. 1, system 100 includes an IMD 110, leads 130A and 130B, and external programmer 150 shown in conjunction with a patient 105, who is ordinarily a human patient, but may be a non-human patient, such as an animal. In the example of FIG. 1, IMD 110 is an implantable electrical stimulator that is configured to generate and deliver electrical stimulation therapy to patient 105 via one or more electrodes of electrodes of leads 130A and/or 130B (collectively, “leads 130”), e.g., for relief of chronic pain or other symptoms. In other examples, IMD 110 may be coupled to a single lead carrying multiple electrodes, to more than two leads each carrying multiple electrodes, or to no leads where the electrodes are disposed on the housing of the IMD. In some examples, the stimulation signals, or pulses, may be configured to elicit detectable evoked response signals that IMD 110 may use to determine the posture state occupied by patient 105 and/or determine how to adjust one or more parameters that define stimulation therapy. IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless.

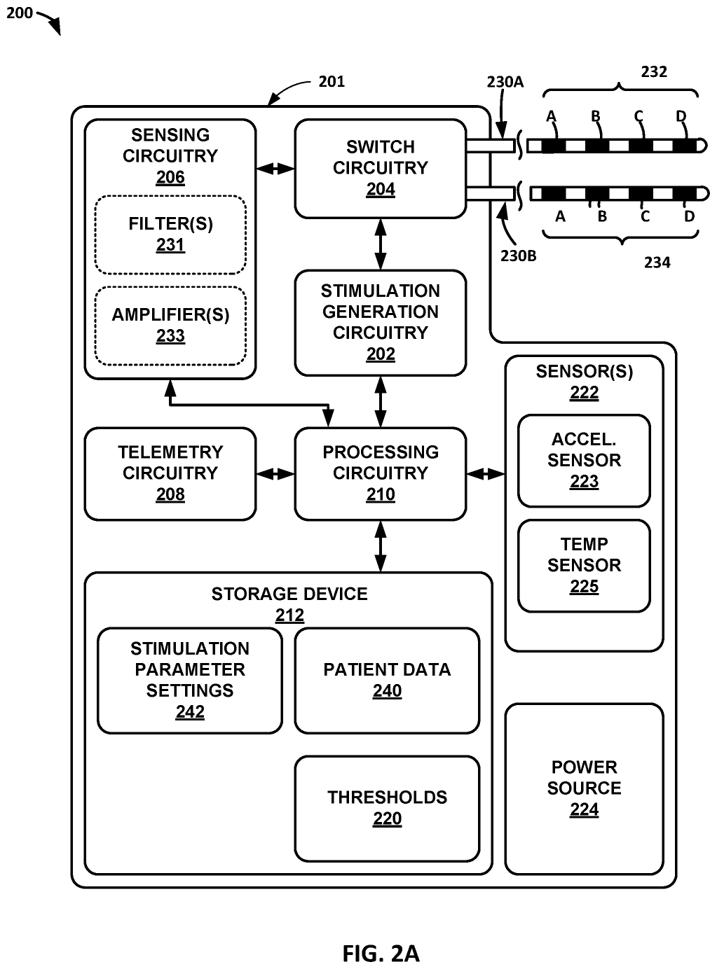

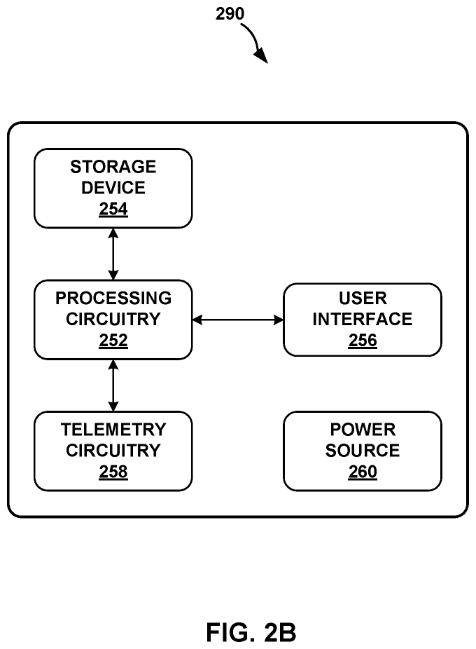

IMD 110 may be constructed of any polymer, metal, or composite material sufficient to house the components of IMD 110 (e.g., components illustrated in FIG. 2A) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge a rechargeable power source resident in IMD 110. In some examples, IMD 110 may be externally powered rather than internally powered, or may be both internally powered and externally powered (e.g., IMD 110 may have a primary power source and a backup power source).

Electrical stimulation energy, which may be constant current or constant voltage-based pulses, for example, is delivered from IMD 110 to one or more target tissue sites of patient 105 via one or more electrodes (not shown) of implantable leads 130. Such an electrical stimulation signal may include active recharge, passive recharge, or hybrid active/passive recharge phases to maintain net charge neutrality. In the example of FIG. 1, leads 130 carry electrodes that are placed adjacent to the target tissue of spinal cord 120. One or more of the electrodes may be disposed at a distal tip of a lead 130 and/or at other positions at intermediate points along the lead. Leads 130 may be implanted and coupled to IMD 110. The electrodes may transfer electrical stimulation generated by an electrical stimulation generator (e.g., stimulation generation circuitry) in IMD 110 to tissue of patient 105. Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The electrodes of leads 130 may be electrode pads on a paddle lead, such as a 5-6-5 lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having eight ring electrodes along the axial length of the lead. In another example, the electrodes are segmented rings arranged in a linear fashion along the axial length of the lead and at the periphery of the lead.

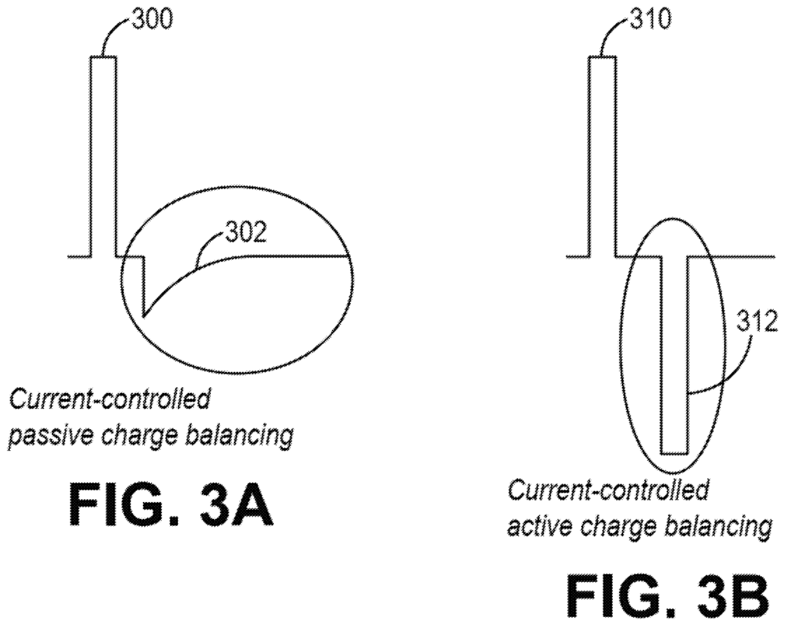

The stimulation parameter set of a therapy stimulation program that defines the stimulation signal of electrical stimulation therapy by IMD 110 through the electrodes of leads 130 may include information identifying which electrodes have been selected for delivery of stimulation according to a stimulation program, the polarities of the selected electrodes, e.g., the electrode combination for the program, voltage or current amplitude, pulse frequency, pulse width, pulse shape of stimulation delivered by the electrodes, a number of interleaved pulses, passive recharge parameters, active recharge parameters, etc. These stimulation parameter values that make up the stimulation parameter set that defines the stimulation signal may be predetermined parameter values defined by a user and/or automatically determined by system 100 based on one or more factors or user input. Active recharge and passive recharge are discussed in more detail with respect to FIGS. 3A-3B below.

As discussed above IMD 110 may analyze a sensed evoked response signal and adjust stimulation recharge parameters of the stimulation signal based on one or more characteristics of one or more artifacts in the sensed evoked response signal. IMD 110 perform such analysis periodically, after each stimulation pulse, on a varied time scale, in response to a clinician or patient request to do so, in response to changing other stimulation parameters or stimulation program, or the like. In some examples, rather than IMD 110 sensing the evoked response signal, one or more external electrodes may sense the evoked response signal. For example, the one or more external electrodes may be coupled to external programmer (via a wired or wireless link) which may communicate the sensed evoked response signal to IMD 110 or may determine whether and how to adjust stimulation recharge parameters of the stimulation signal and communicate how to adjust stimulation recharge parameters to IMD 110.

Although FIG. 1 is directed to SCS therapy, e.g., used to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of DBS, PNS, PNFS, CS, pelvic floor stimulation, gastrointestinal stimulation, or any other stimulation therapy capable of treating a condition of patient 105.

In some examples, lead 130 includes one or more sensors configured to allow IMD 110 to monitor one or more parameters of patient 105, such as patient posture, activity, pressure, temperature, or other characteristics. The one or more sensors may be provided in addition to, or in place of, therapy delivery by lead 130. In some examples, IMD 110 may analyze the sensed evoked response signal to determine whether to adjust stimulation recharge parameters in response to a change in patient posture or activity.

IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the electrical stimulation pulses or continuous waveforms may include an active recharge phase which may be of an opposite polarity to a therapy component to balance out a charge. In other examples, the electrical stimulation pulses may include a passive recharge phase during which no charge is delivered, but built up charge is passively removed. In some examples, the target tissue includes nerves, smooth muscle, or skeletal muscle of the anatomy of patient 105. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

IMD 110 is configured to generate and deliver electrical stimulation therapy to a target stimulation site within patient 105 via the electrodes of leads 130 to patient 105 according to one or more therapy stimulation programs. A therapy stimulation program defines values for one or more stimulation parameters (e.g., a stimulation parameter set) that define an aspect of the therapy delivered by IMD 110 according to that program. For example, a therapy stimulation program that controls delivery of stimulation by IMD 110 in the form of pulses may define values for voltage or current pulse amplitude, pulse width, pulse rate (e.g., pulse frequency), electrode combination, pulse shape, number of interleaved pulses, passive recharge parameters, active recharge parameters, etc. for the stimulation signal delivered by IMD 110 according to that program. Such a therapy stimulation program may include an active recharge phase, a passive recharge phase, or both an active recharge component and a passive recharge phase (e.g., hybrid active/passive recharge).

Furthermore, IMD 110 may be configured to deliver stimulation to patient 105 via a combination of electrodes of leads 130, alone or in combination with an electrode carried by or defined by an outer housing of IMD 110 in order to detect evoked response signals. The tissue targeted by the stimulation may be the same or similar tissue targeted by the electrical stimulation therapy, but IMD 110 may deliver stimulation pulses for evoked response signal detection via the same, at least some of the same, or different electrodes.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from external programmer 150 to control stimulation, such as electrical stimulation therapy. For example, external programmer 150 may transmit therapy stimulation programs, stimulation parameter adjustments (including recharge adjustments), therapy stimulation program selections, evoked response program selections, user input, or other information to control the operation of IMD 110, e.g., by wireless telemetry or wired connection.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy (including stimulation recharge parameters), when a patient perceives stimulation being delivered, or when a patient terminates stimulation due to comfort level. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

As described herein, information may be transmitted between external programmer 150 and IMD 110. Therefore, IMD 110 and external programmer 150 may communicate via wireless communication using any techniques known in the art. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

In some examples, IMD 110, in response to commands from external programmer 150, delivers electrical stimulation therapy according to a plurality of therapy stimulation programs to a target tissue site of the spinal cord 120 of patient 105 via electrodes (not depicted in FIG. 1) on leads 130. In some examples, IMD 110 modifies therapy stimulation programs as therapy needs of patient 105 evolve over time. For example, the modification of the therapy stimulation programs may cause the adjustment of at least one stimulation parameter of the stimulation signal. IMD 110 may also modify or adjust stimulation recharge parameters. When patient 105 receives the same therapy for an extended period, the efficacy of the therapy may be reduced. In some cases, stimulation parameters of the stimulation signal may be automatically updated. In some examples, IMD 110 may detect evoked response signals from the stimulation signal delivered to patient 105 for the purpose of modifying therapy delivered to the patient.

In some examples, efficacy of electrical stimulation therapy may be indicated by one or more characteristics of an action potential that is evoked by a stimulation signal delivered by IMD 110, for example by determining an estimated neural response using a characteristic value of the evoked response signal. Electrical stimulation therapy delivery by leads 130 of IMD 110 may cause neurons within the target tissue to evoke a compound action potential that travels up and down the target tissue, eventually arriving at sensing electrodes of IMD 110. Furthermore, stimulation pulses may also elicit at least one evoked response signal, and evoked responses to stimulation may also be a surrogate for the effectiveness of the therapy and/or the intensity perceived by the patient. The amount of action potentials (e.g., number of neurons propagating action potential signals) that are evoked may be based on the various stimulation parameters of the electrical stimulation signal such as stimulation electrode combination, stimulation electrode polarity, amplitude, pulse width, frequency, pulse shape (e.g., slew rate or slope at the beginning and/or end of the pulse), number of interleaved pulses, passive recharge parameters, active recharge parameters, etc. The slew rate or slope may define the rate of change of the voltage and/or current amplitude of the pulse at the beginning and/or end of each pulse or each phase within the pulse. For example, a very high slew rate or slope indicates a steep or even near vertical edge of the pulse, and a low slew rate or slope indicates a longer ramp up (or ramp down) in the amplitude of the pulse. In some examples, these parameters contribute to an intensity of the electrical stimulation. In addition, a characteristic of the evoked response signal (e.g., an amplitude) may change based on the distance between the stimulation electrodes and the nerves subject to the electrical field produced by the delivered control stimulation pulses.

During delivery of control stimulation pulses defined by one or more evoked response test stimulation programs, IMD 110, via two or more electrodes interposed on leads 130, senses electrical potentials of tissue of the spinal cord 120 of patient 105 to measure the electrical activity of the tissue. IMD 110 senses evoked response from the target tissue of patient 105, e.g., with sensing electrodes on one or more leads 130 and associated sensing circuitry. In some examples, IMD 110 receives a signal indicative of the evoked response from one or more sensors, e.g., one or more electrodes and circuitry, internal or external to patient 105. Such an example signal may include a signal indicating an evoked response of the tissue of patient 105.

In the example of FIG. 1, IMD 110 is described as performing a plurality of processing and computing functions. However, external programmer 150 instead may perform one, several, or all of these functions. In this alternative example, IMD 110 functions to relay sensed signals to external programmer 150 for analysis, and external programmer 150 transmits instructions to IMD 110 to adjust the one or more stimulation parameters defining the electrical stimulation therapy based on analysis of the sensed signals. For example, IMD 110 may relay the sensed signal indicative of an evoked response to external programmer 150. External programmer 150 may compare the parameter value of the evoked response to the target evoked response characteristic value relative to an estimated neural response, and in response to the comparison, external programmer 150 may instruct IMD 110 to adjust one or more stimulation parameters that define the electrical stimulation signal delivered to patient 105.

The stimulation parameters and the target evoked response characteristic may be initially set at the clinic, but may be set and/or adjusted at home by patient 105. For example, the target evoked response characteristics may be changed to match, be a fraction of, or a multiplier of, a stimulation threshold. In some examples, target evoked response characteristics may be specific to respective different posture states of the patient. Once the target evoked response characteristic values are set, the parameter values may be automatically adjusted to maintain consistent volume of neural activation and consistent perception of therapy for the patient. The ability to change the stimulation parameter values may also allow the therapy to have long term efficacy, with the ability to keep the intensity of the stimulation (e.g., as indicated by the evoked response) consistent by comparing the measured evoked response values to the target evoked response characteristic value. In addition, or alternatively, to maintaining stimulation intensity, IMD 110 may monitor the characteristic values of the evoked response signals to limit one or more parameter values that define the stimulation signal. IMD 110 may perform these changes without intervention by a physician or patient 105. In this manner, IMD 110 may deliver closed loop stimulation therapy.

In some examples, the system changes the target evoked response characteristic value over a period of time, such as according to a change to a stimulation threshold (e.g., a perception threshold or detection threshold). The system may be programmed to change the target evoked response characteristic in order to adjust the intensity of stimulation pulses to provide varying sensations to the patient (e.g., increase or decrease the volume of neural activation). Although the system may change the target evoked response characteristic value, received evoked response signals may still be used by the system to adjust one or more parameter values of the stimulation pulse in order to meet the target evoked response characteristic value.

One or more devices within system 100, such as IMD 110 and/or external programmer 150, may perform various functions as described herein. For example, IMD 110 may include stimulation generation circuitry configured to generate a stimulation signal, according to a set of stimulation parameters, to be delivered to anatomy of a patient via a stimulation electrode configuration from a plurality of electrodes. IMD 110 may include sensing circuitry configured to sense an evoked response signal responsive to the stimulation signal. IMD 110 may include processing circuitry communicatively coupled to the stimulation generation circuitry and the sensing circuitry. As used herein, the processing circuitry being communicatively coupled to the stimulation generation circuitry and the sensing circuitry means that the processing circuitry is coupled to the stimulation generation circuitry and the sensing circuitry in a manner in which processing circuitry may communicate with the stimulation generation circuitry and the sensing circuitry. For example, the processing circuitry may control the stimulation generation circuitry and/or the sensing circuitry. For example, the processing circuitry may also receive signals from the sensing circuitry. The processing circuitry may be being configured to control the stimulation generation circuitry to generate the stimulation signal and receive, from the sensing circuitry, the sensed evoked response signal. The processing circuitry may control the stimulation generation circuitry to generate the first stimulation signal having the first stimulation recharge parameter for delivery to the target anatomy. The processing circuitry may receive from the sensing circuitry the sensed evoked response signal. The processing circuitry may analyze the sensed evoked response signal for one or more artifacts and adjust, based on the one or more artifacts, the first stimulation recharge parameter to determine a second stimulation recharge parameter. The processing circuitry may control the stimulation generation circuitry to generate a second stimulation signal having the second stimulation recharge parameter for delivery to the target anatomy. For example, IMD 110 may change a stimulation recharge parameter based on the sensed evoked response signal.

In some examples, IMD 110 may include the stimulation generation circuitry, the sensing circuitry, and the processing circuitry. However, in other examples, one or more additional devices may be part of the system that performs the functions described herein. For example, IMD 110 may include the stimulation generation circuitry and the sensing circuitry, but external programmer 150 or other external device may include the processing circuitry that at least analyzes the sensed evoked response signal. IMD 110 may transmit the sensed evoked response signals, or data representing the evoked response signal, to external programmer 150, for example. Therefore, the processes described herein may be performed by multiple devices in a distributed system. In some examples, system 100 may include one or more electrodes that deliver and/or sense electrical signals. Such electrodes may be configured to deliver stimulation and/or sense the evoked response signals. In some examples, the same electrodes may be configured to sense signals representative of transient movements of the patient. In other examples, other sensors, such as accelerometers, gyroscopes, or other movement sensors may be configured to sense movement of the patient that indicates the patient may have transitioned to a different posture state.

Although in one example IMD 110 takes the form of an SCS device, in other examples, IMD 110 takes the form of any combination of DBS devices, peripheral nerve stimulators, muscle stimulators, implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine stimulation thresholds (e.g., perception thresholds and detection thresholds) associated any one of the aforementioned IMDs and then use a stimulation threshold to inform the intensity (e.g., stimulation levels) of therapy.

FIG. 2A is a block diagram illustrating an example combination of components of an IMD 200, in accordance with one or more techniques of this disclosure. IMD 200 may be an example of IMD 110 of FIG. 1. In the example shown in FIG. 2A, IMD 200 includes stimulation generation circuitry 202, switch circuitry 204, sensing circuitry 206, telemetry circuitry 208, processing circuitry 210, storage device 212, sensor(s) 222, and power source 224.

In the example shown in FIG. 2A, storage device 212 stores patient data 240, stimulation parameter settings 242, and various thresholds discussed in this disclosure in separate memories within storage device 212 or separate areas within storage device 212. Patient data 240 may include parameter values, target evoked response characteristic values, or other information specific to the patient. In some examples, stimulation parameter settings 242 may include stimulation parameter values for respective different stimulation programs selectable by the clinician or patient for therapy. In this manner, each stored therapy stimulation program, or set of stimulation parameter values, of stimulation parameter settings 242 defines values for a set of electrical stimulation parameters (e.g., a stimulation parameter set), such as a stimulation electrode combination, electrode polarity, current or voltage amplitude, pulse width, pulse rate, pulse shape, duty cycle, number of interleaved pulses, passive recharge parameters, active recharge parameters, etc.

Accordingly, in some examples, stimulation generation circuitry 202 generates electrical stimulation signals in accordance with the electrical stimulation parameters noted above. Other ranges of stimulation parameter values may also be useful and may depend on the target stimulation site within patient 105. Stimulation signals may be of any form, such as continuous-time signals (e.g., sine waves) or the like. Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232, 234, or directed sensed signals from one or more of electrodes 232, 234 to sensing circuitry 206. In other examples, stimulation generation circuitry 202 and/or sensing circuitry 206 may include sensing circuitry to direct signals to and/or from one or more of electrodes 232, 234, which may or may not also include switch circuitry 204. While not shown in FIG. 2A, in some examples at least one external reference electrode may be used to sense electrode potentials to detect the ECAP and artifact. In some examples, switch circuitry 204 may include DC blocking capacitors (not shown in FIG. 2A) which may be coupled a low impedance reference (e.g., ground) during a passive recharge phase.

Sensing circuitry 206 is configured to monitor signals from any combination of electrodes 232, 234. In some examples, sensing circuitry 206 includes one or more amplifiers, filters, and analog-to-digital converters. For example, amplifier(s) 233 of sensing circuitry 206 may amplify a sensed evoked response signal and/or filter(s) 231 of sensing circuitry 206 may filter a sensed evoked response signal which may be used to remove or reduce the impact of artifacts on a sensed evoked response signal. Sensing circuitry 206 may be used to sense physiological signals, such as evoked response signals. In some examples, sensing circuitry 206 detects evoked response from a particular combination of electrodes 232, 234. In some cases, the particular combination of electrodes for sensing evoked response includes different electrodes than a set of electrodes of electrodes 232, 234 used to deliver stimulation pulses. Alternatively, in other cases, the particular combination of electrodes used for sensing evoked response includes at least one of the same electrodes as a set of electrodes used to deliver stimulation pulses to patient 105. Sensing circuitry 206 may provide signals to an analog-to-digital converter (not shown), for conversion into a digital signal for processing, analysis, storage, or output by processing circuitry 210.

Telemetry circuitry 208 supports wireless communication between IMD 200 and an external programmer (not shown in FIG. 2A) or another computing device under the control of processing circuitry 210. Processing circuitry 210 of IMD 200 may receive, as updates to programs, values for various stimulation parameters such as amplitude and electrode combination, from the external programmer via telemetry circuitry 208. Processing circuitry 210 may store updates to the stimulation parameter settings 242 or any other data in storage device 212. Telemetry circuitry 208 in IMD 200, as well as telemetry circuitry in other devices and systems described herein, such as the external programmer, may accomplish communication by radiofrequency (RF) communication techniques. In addition, telemetry circuitry 208 may communicate with an external medical device programmer (not shown in FIG. 2A) via proximal inductive interaction of IMD 200 with the external programmer. The external programmer may be one example of external programmer 150 of FIG. 1. Accordingly, telemetry circuitry 208 may send information to the external programmer on a continuous basis, at periodic intervals, or upon request from IMD 110 or the external programmer.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 controls stimulation generation circuitry 202 to generate stimulation signals according to stimulation parameter settings 242 and any other instructions stored in storage device 212 to apply stimulation parameter values specified by one or more of programs, such as electrode combination, electrode polarity, amplitude, pulse width, pulse rate, pulse shape, number of interleaved pulses, passive recharge parameters, etc., of each of the stimulation signals.

In the example shown in FIG. 2A, lead 230A is shown having a set of electrodes 232 that includes electrodes 232A-232D, and lead 230B is shown having a set of electrodes 234 includes electrodes 234A-234D. However, lead 230A and lead 230 B may have any number of electrodes, such as 8 electrodes or 16 electrodes. In other examples, a single lead may be coupled to IMD 200 which may include any number of electrodes, such as include 8 electrodes or 16 electrodes along a single axial length of the lead.

Processing circuitry 210 also controls stimulation generation circuitry 202 to generate and apply the stimulation signals to selected combinations of electrodes 232, 234. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord of the patient (not shown in FIG. 2A) with selected electrodes 232, 234.

In other examples, however, stimulation generation circuitry 202 does not include a switch circuit and switch circuitry 204 does not interface between stimulation generation circuitry 202 and electrodes 232, 234. In these examples, stimulation generation circuitry 202 includes a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via switch circuitry 204 and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 206 is incorporated into a common housing with stimulation generation circuitry 202 and processing circuitry 210 in FIG. 2A, in other examples, sensing circuitry 206 may be in a separate housing from IMD 200 and may communicate with processing circuitry 210 via wired or wireless communication techniques. In some examples, one or more of electrodes 232 and 234 are suitable for sensing the evoked responses. For instance, electrodes 232 and 234 may sense the voltage amplitude of a portion of the evoked response signals, where the sensed voltage amplitude, such as the voltage difference between features within the signal, is a characteristic the evoked response signal.

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), ferroelectric random access memories (FRAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store patient data 240, stimulation parameter settings 242, and thresholds 220. In some examples, IMD 200 may store information associated with how IMD 200 has adjusted stimulation recharge parameters in the past in stimulation parameter settings 242. In some examples, IMD 200 may adjust stimulation recharge parameters based on previous adjustments.

Sensor(s) 222 may include one or more sensing elements that sense values of a respective patient parameter, such as posture state. As described, electrodes 232 and 234 may be the electrodes that sense the characteristic value of the evoked response signal. Sensor(s) 222 may include one or more accelerometers, optical sensors, chemical sensors, temperature sensors, pressure sensors, or any other types of sensors. Sensor(s) 222 may output patient parameter values that may be used as feedback to control delivery of therapy. For example, sensor(s) 222 may indicate patient activity, and processing circuitry 210 may increase the frequency of control pulses and evoked response sensing in response to detecting increased patient activity. In one example, processing circuitry 210 may initiate control pulses and corresponding evoked response sensing in response to a signal from sensor(s) 222 indicating that patient activity has exceeded an activity threshold. Conversely, processing circuitry 210 may decrease the frequency of control pulses and evoked response sensing in response to detecting decreased patient activity. For example, in response to sensor(s) 222 no longer indicating that the sensed patient activity exceeds a threshold, processing circuitry 210 may suspend or stop delivery of control pulses and evoked response sensing. In this manner, processing circuitry 210 may dynamically deliver control pulses and sense evoked response signals based on patient activity to reduce power consumption of the system when the electrode-to-neuron distance is not likely to change and increase system response to evoked response changes when electrode-to-neuron distance is likely to change. IMD 200 may include additional sensors within the housing of IMD 200 and/or coupled via one of leads 230 or other leads. In addition, IMD 200 may receive sensor signals wirelessly from remote sensors via telemetry circuitry 208, for example. In some examples, one or more of these remote sensors may be external to patient (e.g., carried on the external surface of the skin, attached to clothing, or otherwise positioned external to patient 105). In some examples, signals from sensor(s) 222 indicate a position, body state, or posture (e.g., sleeping, awake, sitting, standing, or the like), and processing circuitry 210 may select target evoked response characteristic values according to the indicated position or body state.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries, one or more capacitors, or the like.

FIG. 2B is a block diagram illustrating an example combination of components of an example external programmer 290. External programmer 290 may be an example of external programmer 150 of FIG. 1. Although external programmer 290 may generally be described as a hand-held device, external programmer 290 may be a larger portable device or a more stationary device. In addition, in other examples, external programmer 290 may be included as part of an external charging device or include the functionality of an external charging device. As illustrated in FIG. 2B, external programmer 290 may include processing circuitry 252, storage device 254, user interface 256, telemetry circuitry 258, and power source 260. Storage device 254 may store instructions that, when executed by processing circuitry 252, cause processing circuitry 252 and external programmer 290 to provide the functionality ascribed to external programmer 290 throughout this disclosure. Each of these components, circuitry, or modules, may include electrical circuitry that is configured to perform some, or all of the functionality described herein. For example, processing circuitry 252 may include processing circuitry configured to perform the processes discussed with respect to processing circuitry 252.

In general, external programmer 290 includes any suitable arrangement of hardware, alone or in combination with software and/or firmware, to perform the techniques attributed to external programmer 290, and processing circuitry 352, user interface 356, and telemetry circuitry 358 of external programmer 290. In various examples, external programmer 290 may include one or more processors, such as one or more microprocessors, DSPs, ASICs, FPGAs, or any other equivalent integrated or discrete logic circuitry, as well as any combinations of such components. External programmer 290 also, in various examples, may include a storage device 254, such as RAM, ROM, PROM, EPROM, EEPROM, flash memory, a hard disk, a CD-ROM, including executable instructions for causing the one or more processors to perform the actions attributed to them. Moreover, although processing circuitry 252 and telemetry circuitry 258 are described as separate modules, in some examples, processing circuitry 252 and telemetry circuitry 258 are functionally integrated. In some examples, processing circuitry 252 and telemetry circuitry 258 correspond to individual hardware units, such as ASICs, DSPs, FPGAs, or other hardware units.

Storage device 254 (e.g., a storage device) may store instructions that, when executed by processing circuitry 252, cause processing circuitry 252 and external programmer 290 to provide the functionality ascribed to external programmer 290 throughout this disclosure. For example, storage device 254 may include instructions that cause processing circuitry 252 to obtain a parameter set from memory, select a spatial electrode pattern, or receive a user input and send a corresponding command to IMD 200, or instructions for any other functionality. In addition, storage device 254 may include a plurality of programs, where each program includes a stimulation parameter set that defines therapy stimulation or control stimulation, including active stimulation recharge parameter(s) and/or passive stimulation recharge parameter(s). Storage device 254 may also store data received from a medical device (e.g., IMD 110). For example, storage device 254 may store evoked response related data sensed by sensing circuitry of the medical device, and storage device 254 may also store data from one or more sensors of the medical device.

User interface 256 may include a button or keypad, lights, a speaker for voice commands, a display, such as a liquid crystal (LCD), light-emitting diode (LED), or organic light-emitting diode (OLED). In some examples the display includes a touch screen. User interface 256 may be configured to display any information related to the delivery of electrical stimulation, identified posture states, sensed patient parameter values, or any other such information. User interface 256 may also receive user input (e.g., indication of when the patient perceives a stimulation pulse) via user interface 256. The input may be, for example, in the form of pressing a button on a keypad or selecting an icon from a touch screen. The input may request starting or stopping electrical stimulation, the input may request different stimulation parameters (e.g., to change a stimulation program), or the input may request some other change to the delivery of electrical stimulation or sensing.

Telemetry circuitry 258 may support wireless communication between the IMD 200 and external programmer 290 under the control of processing circuitry 252. Telemetry circuitry 258 may also be configured to communicate with another computing device via wireless communication techniques, or direct communication through a wired connection. In some examples, telemetry circuitry 258 provides wireless communication via an RF or proximal inductive medium. In some examples, telemetry circuitry 258 includes an antenna, which may take on a variety of forms, such as an internal or external antenna.

Examples of local wireless communication techniques that may be employed to facilitate communication between external programmer 290 and IMD 110 include RF communication according to the 802.11 or Bluetooth® specification sets or other standard or proprietary telemetry protocols. In this manner, other external devices may be capable of communicating with external programmer 290 without needing to establish a secure wireless connection. As described herein, telemetry circuitry 258 may be configured to transmit a spatial electrode movement pattern or other stimulation parameter values to IMD 110 for delivery of electrical stimulation therapy. Although IMD 110 may determine characteristic values for evoked response signals and control the adjustment of stimulation parameter values or sensing parameters, in some examples, programmer 290 may perform these tasks alone or together with IMD 110 in a distributed function.

In some examples, selection of stimulation parameters or therapy stimulation programs and/or sensing parameters are transmitted to the medical device for delivery of stimulation to a patient (e.g., patient 105 of FIG. 1) and sensing of evoked response signals. In other examples, the therapy may include medication, activities, or other instructions that patient 105 must perform themselves or a caregiver perform for patient 105. In some examples, external programmer 290 provides visual, audible, and/or tactile notifications that indicate there are new instructions. External programmer 290 requires receiving user input acknowledging that the instructions have been completed in some examples.

User interface 256 of external programmer 290 may also be configured to receive an indication from a clinician instructing a processor of the medical device to update one or more therapy stimulation programs or to update the target characteristic values for evoked response signals. Updating therapy stimulation programs and target characteristic values may include changing one or more parameters of the stimulation pulses delivered by the medical device according to the programs, such as amplitude, pulse width, frequency, pulse shape of the pulses and/or control pulses, electrode combinations, electrode polarity, number of interleaved pulses, passive recharge parameters, active recharge parameters, etc. User interface 256 may also receive instructions from the clinician commanding any electrical stimulation, including therapy stimulation and control stimulation to commence or to cease.

Power source 260 is configured to deliver operating power to the components of external programmer 290. Power source 260 may include a battery and a power generation circuit to produce the operating power. In some examples, the battery is rechargeable to allow extended operation. Recharging may be accomplished by electrically coupling power source 260 to a cradle or plug that is connected to an alternating current (AC) outlet. In addition, recharging may be accomplished through proximal inductive interaction between an external charger and an inductive charging coil within external programmer 290. In other examples, traditional batteries (e.g., nickel cadmium or lithium ion batteries) may be used. In addition, external programmer 290 may be directly coupled to an alternating current outlet to operate.

The architecture of external programmer 290 illustrated in FIG. 2B is shown as an example. The techniques as set forth in this disclosure may be implemented in the example external programmer 290 of FIG. 2B, as well as other types of systems not described specifically herein. Nothing in this disclosure should be construed so as to limit the techniques of this disclosure to the example architecture illustrated by FIG. 2B.

FIGS. 3A-3B are conceptual diagrams illustrating an example passive charge balancing waveform and an example active charge balancing waveform, respectively. While the waveforms of FIGS. 3A-3B are generally charge balanced, the techniques of this disclosure may also apply to waveforms that are intentionally not charge balanced, for example, when there are no DC blocking capacitors and the net DC charge is within some safety limit. While the waveforms are described with respect to current, in some examples, the waveforms may be voltage waveforms. The biphasic stimulation signal delivered to the target anatomy using IMD 200 (FIG. 2) may include a passive and/or active recharge phase (e.g., charge balancing phase). The intent of this recharge phase is to deliver a charge balanced waveform (e.g., generally maintain net charge neutrality) and prevent charge build up on both DC blocking capacitors of IMD 200, as well as the electrode-tissue interface (e.g., the interface between the electrodes and the tissue proximate to the electrodes).

During passive recharge (FIG. 3A), IMD 110 may passively remove the charge caused by stimulation signal 300 by reconnecting electrode interface via DC blocking capacitors to some low impedance reference. Built-up charge is thereby allowed to passively dissipate 302 between the series combination of resistance and capacitance in a loop.

During active recharge (FIG. 3B), IMD 110 may apply a current of the opposite polarity of the stimulation pulses between stimulating electrodes. The recharge current 312 can be the same in amplitude, duration, and/or shape as the stimulating current 310, or different in amplitude, duration, and/or shape. Overall, however, in some examples, a generally equivalent amount of charge is delivered during the active recharge (e.g., second) phase as is delivered during the stimulating (first) phase of the biphasic stimulation signal.

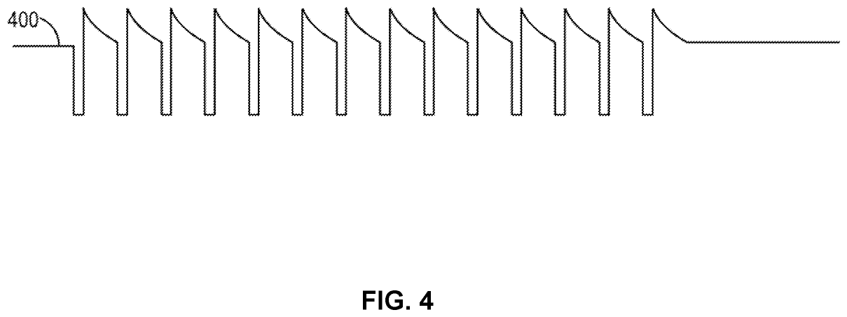

FIG. 4 is a conceptual diagram illustrating an example stimulus waveform having passive recharge balancing. IMD 200 may apply stimulus waveform 400 to a subset of electrodes during the delivery of stimulation therapy. IMD 200 may follow stimulus waveform 400 with sensing activity on either the same or a separate set of electrodes to monitor an evoked neural response.

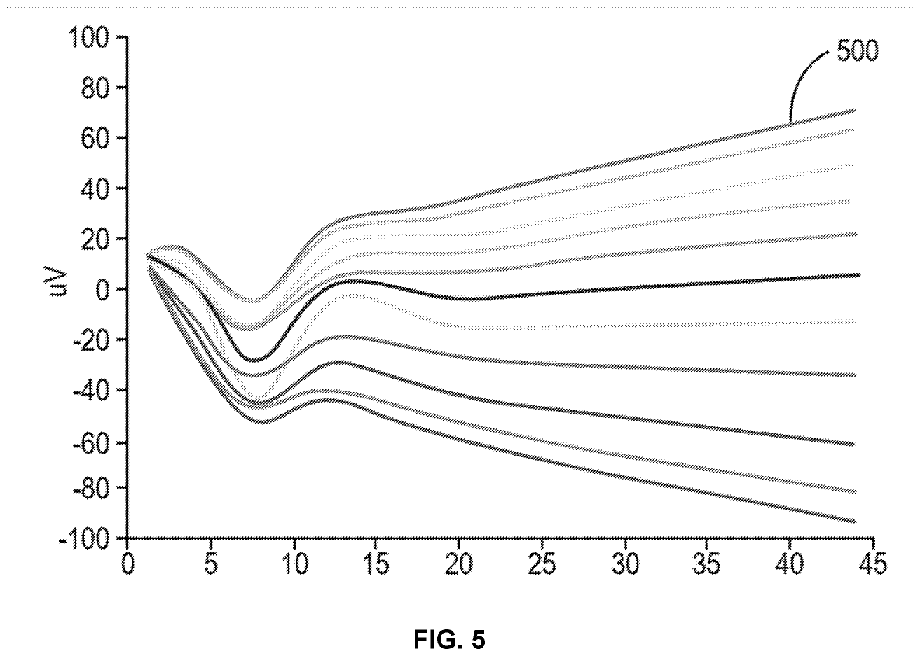

FIG. 5 is a conceptual diagram illustrating examples of stimulation artifacts superimposed on top of an example tissue neural response signal. While stimulus and sensing may be separated spatially and temporally, there is typically a post-stimulus artifact present at the electrode-tissue interface (e.g., where the electrode meets tissue). This artifact, e.g., artifact 500, may become superimposed on top of a neural signal of interest and may even saturate the sensing channel, which, in turn, may result in sub-optimal sensing. In some examples, the effect of the artifact may depend on stimulation parameters such as pulse rate, current or voltage amplitude, pulse width, and/or stimulation/sensing electrode proximity. These limitations may potentially restrict delivered therapy to specific set of electrodes or stimulus parameters, which may be undesirable.

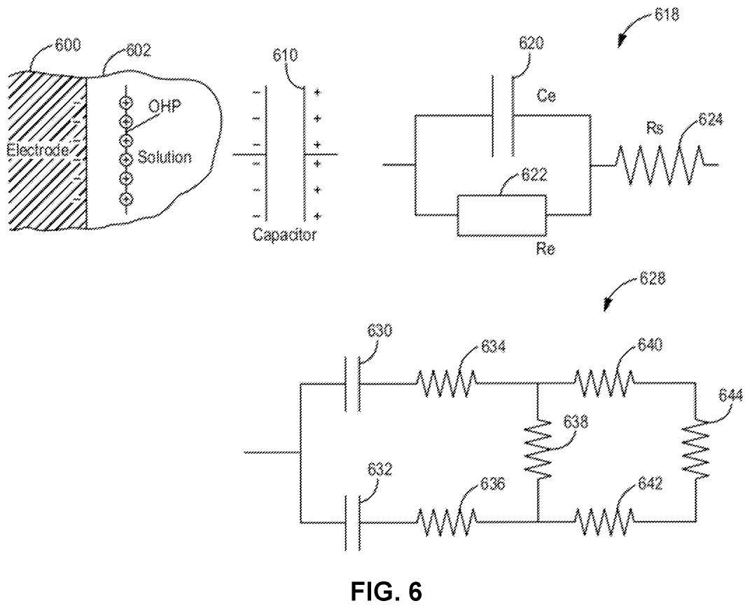

FIG. 6 is a conceptual diagram illustrating example simplified double layer capacitor models for an electrode-tissue interface. FIG. 6 may be used to better understand some sources of post-stimulus artifact(s). The double layer 602 at the electrode 600 interface to surrounding tissue not only resembles a capacitor (e.g., capacitor 610) in structure, but double layer 602 also acts like capacitor 610 (charging and discharging with potential changes). Energy stored in double layer 602 may drive chemical reactions when the applied current is zero.

The electrode double layer 602 current-potential behavior may be modeled as an RC-circuit, for example, RC-circuit 618 including: Ce 620—double layer capacitance (which may account for charge storage in double 602 layer); Re 622—charge transfer resistance (e.g., a voltage dependent parameter that may account for Faradaic redox reactions); and Rs 624—conductor and electrolyte resistance. Additionally or Alternatively, the electrode double layer 602 current-potential behavior may be modeled as RC-circuit 628 having a plurality of capacitors representing double layered capacitances 630 and 632 and a plurality of resistors representing resistances 634, 636, 638, 640, 642, and 644 for an electrode.

A double layer model for the electrode interface applies across the entire range of voltages seen by the electrode i(Ve) profile (e.g., cyclic voltammogram), however additional processes can simultaneously take place at the electrode interface as the potential is moved further in the positive or negative direction away from the electrode open circuit potential (OCP). If electrode 600 is left at a potential positive of the OCP after a stimulus, oxidation may occur at electrode 600 to discharge double layer 602 and the electrode potential may drift negative towards OCP. Conversely, if electrode 600 is left at potential negative of the OCP, reduction will occur to discharge double layer 602 with a subsequent positive potential drift towards OCP. Therefore, it is desirable to keep the electrode potentials within the area of operation that result in reversable electrochemical reactions. This may be done to avoid harmful effects such as tissue damage and electrode dissolution.

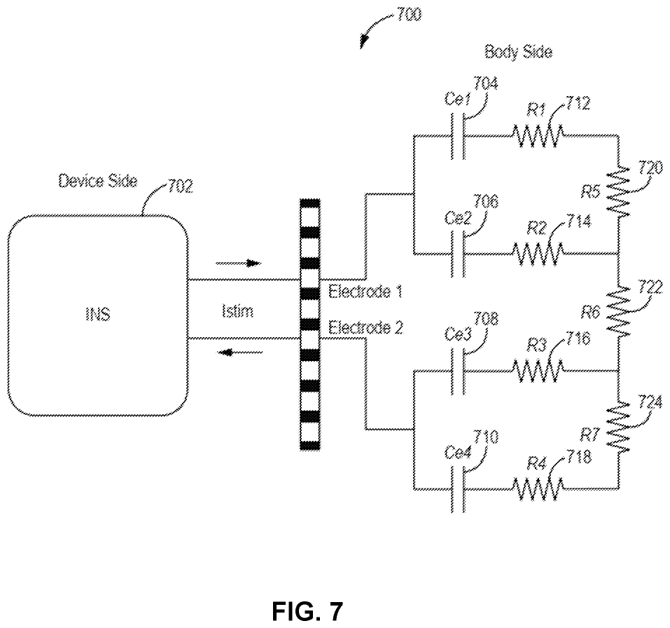

FIG. 7 is a schematic diagram illustrating an example simplified model of implantable neurostimulation device and tissue interface during stimulation. This simplified electrode interface 700 between electrodes of IMD 702 (which may be an example of IMD 110 or IMD 200) and surrounding tissue using a double layer capacitor may be used for each stimulation electrode. For example, for stimulation electrodes, the model may include double layer capacitances (Ce1 704, Ce2 706, Ce3 708, and Ce4 710), and resistances (R1 712, R2 714, R3 716, R4 718, R5 720, R6 722, and R7 724) which may represent an electrode tissue interface load. Note that size of the load and complexity of distributed network may vary for various models and may include a resistive network beyond R5 720-R7 724.

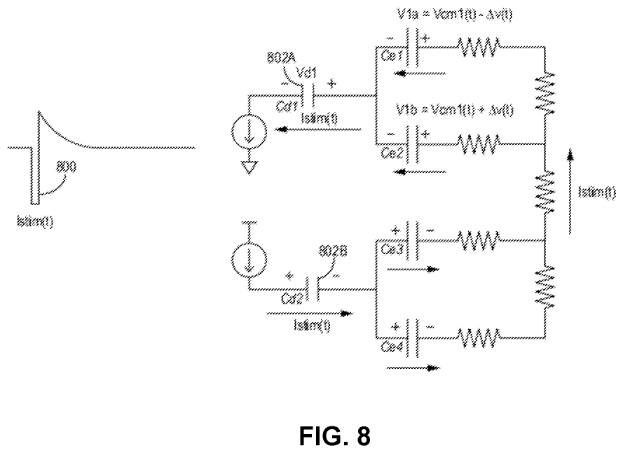

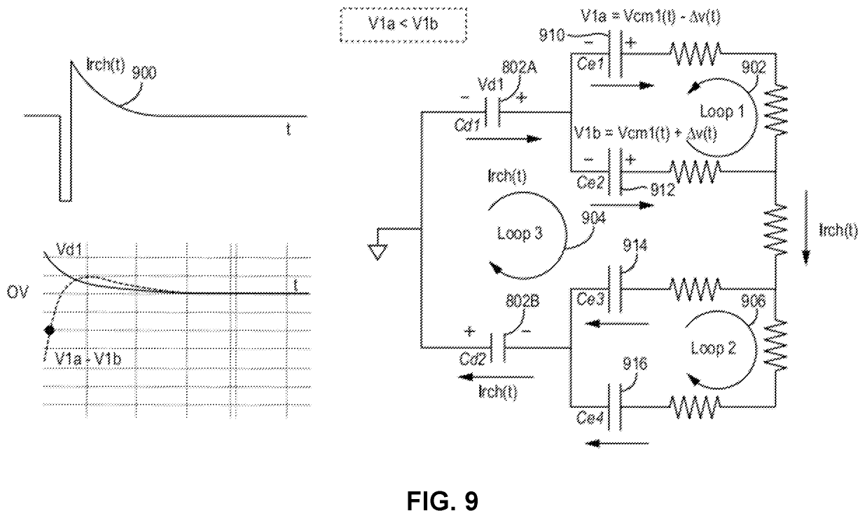

FIG. 8 is a schematic diagram illustrating an example device and electrode charging during a leading phase of a stimulus pulse. During leading phase 800 of the stimulation signal, both the device and electrode double layer capacitance are charged (V1a, V1b) and polarized as shown in FIG. 8. Here, Vcm1 is common voltage potential and Δv is differential voltage potential for distributed capacitance within a same electrode. During a passive recharge phase, the device side of DC blocking capacitors (e.g., capacitors 802A and 802B) may be reconnected to a low impedance reference, such as ground as shown in FIG. 9.

FIG. 9 is a schematic diagram illustrating example device and electrode capacitors during a passive recharge phase of a stimulation signal. In the example of FIG. 9, passive recharge phase 900 is shown and capacitors 802A and 802B (Cd1, Cd2) are coupled to ground on the device side. The main discharge loops can be viewed as follows: Loop 1 902 is a discharge loop that is present at double layer 602 (FIG. 6) because voltage V1b is not equal to V1a. This loop may be controlled by the double layer capacitance formed at the electrode interface along with an electrode electrode-tissue interface load. This loop may be present for each stimulating electrode in some form both during stimulation delivery and passive recharge (e.g., for loop 1 902 and loop 2 906).

Loop 3 904 is a discharge loop that is present for both device capacitors 802A and 802B (Cd1, Cd2) and electrode double layer capacitors 910, 912, 914, and 916 (Ce1, Ce2, Ce3, Ce4) via the electrode tissue interface load if a return path for current to follow (such as during passive recharge operation) is provided on the device side (e.g., IMD 110).

In the presence of an electrode-tissue interface load, Loop 1 902 and Loop 2 906 provide an additional discharge path for double layer capacitors 910, 912, 914, and 916 (Ce1, Ce2, Ce3, Ce4). This is in contrast to device capacitors 802A and 802B (Cd1, Cd2) which do not have this additional discharge path. The intrinsic discharge path associated with capacitors 910, 912, 914, and 916 (Ce1, Ce2, Ce3, Ce4) may result in elimination or reduction of a voltage gradient within each stimulus electrode (differential potential Δv) before the device capacitors 802A and 802B (Cd1, Cd2) are fully discharged.

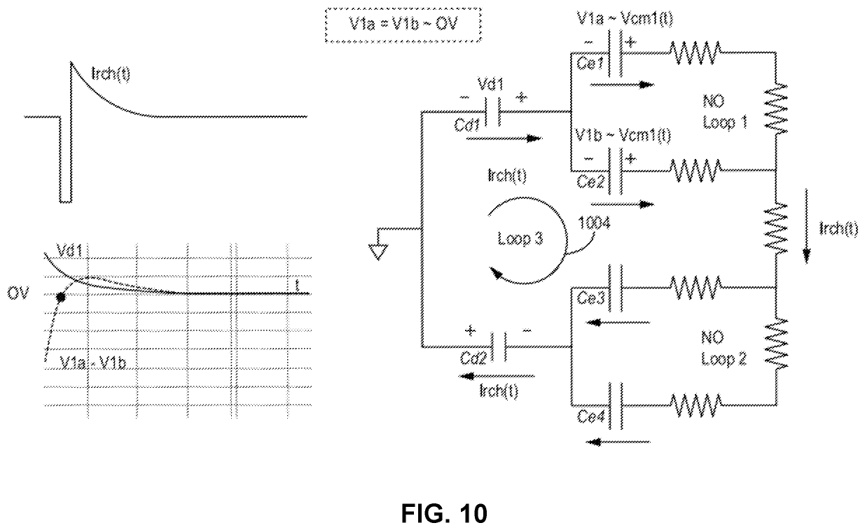

FIG. 10 is a schematic diagram illustrating an example 0 volt crossing on a double layer capacitor during passive recharging. A 0 volt crossing may be defined as a condition at which a voltage gradient within each electrode is minimized (V1a−V1b˜0V). As post stimulus recharge continues, the gradient voltage within an electrode interface continues to change until V1a−V1b˜0V) which results in effectively stopping a discharge loop within the electrodes (Loop 1 902 and Loop 2 906 of FIG. 9). If post stimulus recharge is stopped at this time, a sensing artifact is reduced because there is no extra discharge loop within the electrode interface. Common voltage Vcm1 of one electrode might not be equal common voltage Vcm2 of the other electrode which can result in a different type of sensing artifact through Loop 3 1004. However, this sensing artifact may be a relatively slowly changing artifact compared to the sensing artifact caused by Loop 1 902 and Loop 2 906 and may be removed from a sensing signal by other techniques.

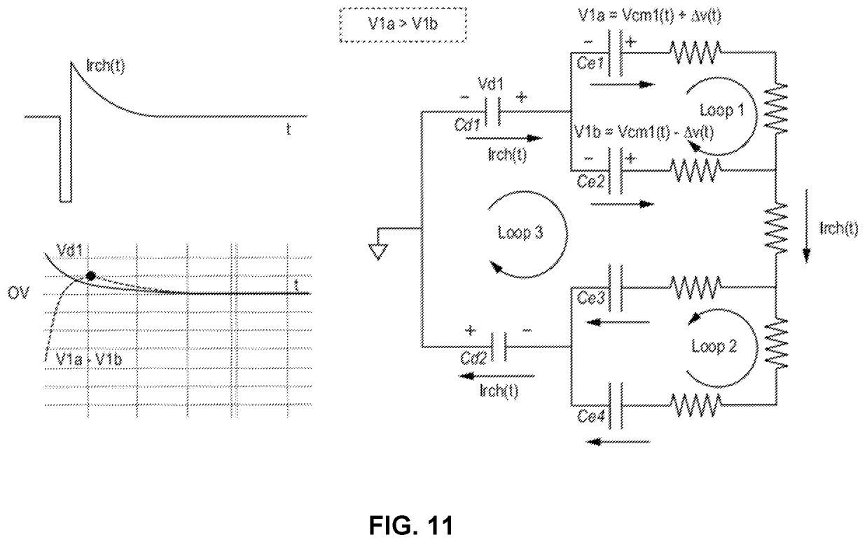

FIG. 11 is a schematic diagram illustrating an example re-polarization of a double layer capacitor. If passive recharge continues beyond this 0 volt crossing point, the double layer capacitance may become re-polarized in the opposite direction as shown in FIG. 11 as V1a voltage may exceed V1b voltage. This manifests as a reversal of the artifact polarity. This electrode re-polarization may occur because the double layer capacitance is discharged when there is voltage still present on device capacitor (Cd). As a result, the device capacitance can repolarize electrode capacitance in the opposite direction from the initial post-stimulation potential across the double layer capacitor. This process may continue until both capacitors may begin discharging together and may approach 0V for long passive recharge durations.

To summarize, the time at which passive recharge is truncated or paused (for example, passive recharge may pause for an amount of time and then resume) impacts the residual charge left on the double layer capacitance. This in turn correlates with the extent of a post stimulus artifact that is sensed. The amount of an artifact may be minimized by either truncating or pausing the passive recharge before the double layer capacitance gets re-polarized, or by extending passive recharge long enough to allow both the device and electrode capacitances to sufficiently discharge towards 0V.

From the electrode perspective, both of these techniques result in an electrode interface closer to the OCP state. The difference between the two techniques is in the residual voltage potential left on the device capacitance. However, keeping passive recharge enabled for a long time may place timing constraints on the stimulation therapy and, in turn, result in limitations placed on a stimulus rate and other stimulus parameters. Passive recharge truncation or pausing at the 0V-crossing may therefore be of interest as an alternative technique to keeping passive recharge for an extended period of time, as well as provide direct control to minimize any artifact during a sensing window. For example, an IMD, like IMD 200, may analyze a sensed evoked response signal and adjust change passive recharge parameter(s) to truncate or pause the passive recharge phase at or near the 0V crossing to minimize an artifact. IMD 200 may truncate or pause the passive recharge phase by disconnecting the device side DC blocking capacitors 802A and 802B from the low impedance (e.g., ground) and/or reconnecting DC blocking capacitor 802A to stimulation generation circuitry 202.

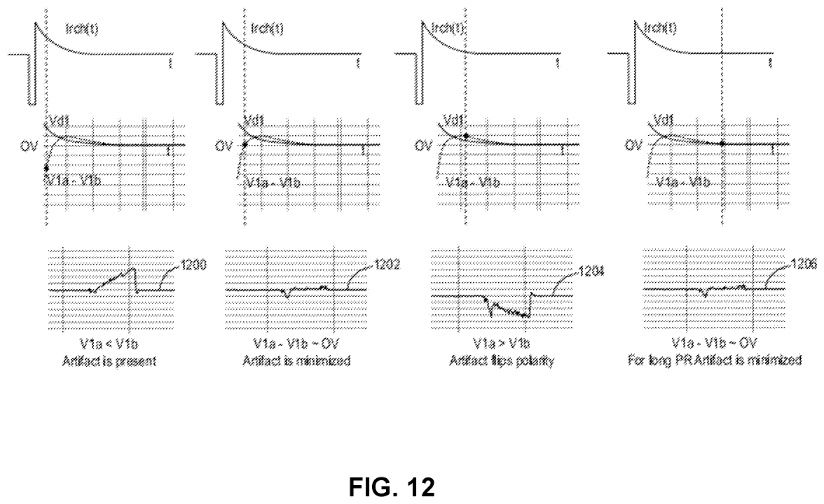

FIG. 12 is a conceptual diagram illustrating example artifact and electrode voltage change depending on when passive recharge stops. As can be seen, during the passive recharge phase, the double layer capacitance is polarized (V1a<V1b) and the artifact is present in signal 1200. At the zero crossing (V1a−V1b˜0V), the artifact is minimized in signal 1202. After the zero crossing, where the double layer capacitance is repolarized, the artifact is present and in an opposite polarity from signal 1200 in signal 1204. If given enough time, the double layer capacitance may discharge back to 0V and the artifact may again be minimized as shown in signal 1206. As such, an IMD using passive recharge may truncate or pause the passive recharge at or near the 0V crossing or may wait a sufficient time period prior to delivering another stimulation pulse such that the double layer capacitance may discharge back to 0V.

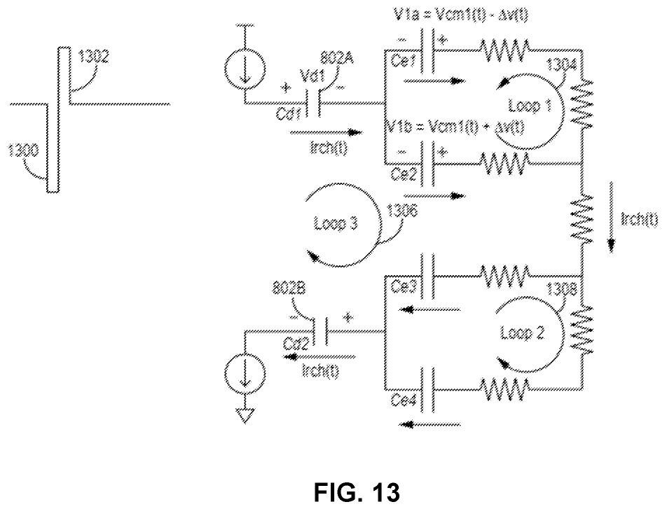

FIG. 13 is a schematic diagram illustrating device and electrode capacitors discharging in an active recharge phase. While the above analysis is focused on passive recharge, the same concepts may hold true for active recharge as well. In this example, an active recharge phase or component 1302 follows stimulation pulse 1300. During active recharge, the device side of the blocking capacitors 802A and 802B (Cd1, Cd2) is reconnected to current sources (or voltage sources) of opposite polarity to stimulation current (or voltage) as shown in FIG. 13. The main discharge loops are the same as described during passive recharge (Loop 1 1304, Loop 2 1308 and Loop 3 1306) and, therefore, double layer capacitance may cross 0V before device capacitance may be fully discharged.

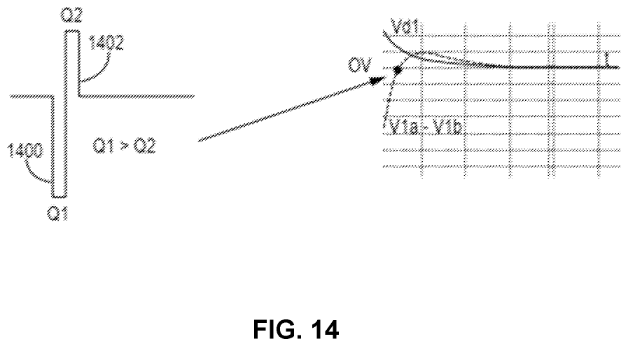

FIG. 14 is a conceptual diagram illustrating 0 volt crossing on a double layer capacitor during active recharging. The amount (e.g., amplitude) of artifact after active recharge may be minimized by adjusting the duration or amplitude of the active recharge phase 1402 such that charge left on the double layer capacitance is minimized. That means the active recharge phase 1402 should have less total charge (e.g., a lower amplitude and/or a shorter duration) compared to the stimulating phase 1400 to account for charge lost by Loop 1 1304 (FIG. 13), as shown in FIG. 14.

Therefore, according to the techniques of this disclosure, IMD 110 may control electrode operating potential by (1) analyzing characteristics of the sensed artifact and, (2) adjusting stimulation recharge parameters in a way that both minimizes or reduces the artifact and optimizes or improves the electrode potential within desired limits of operation.

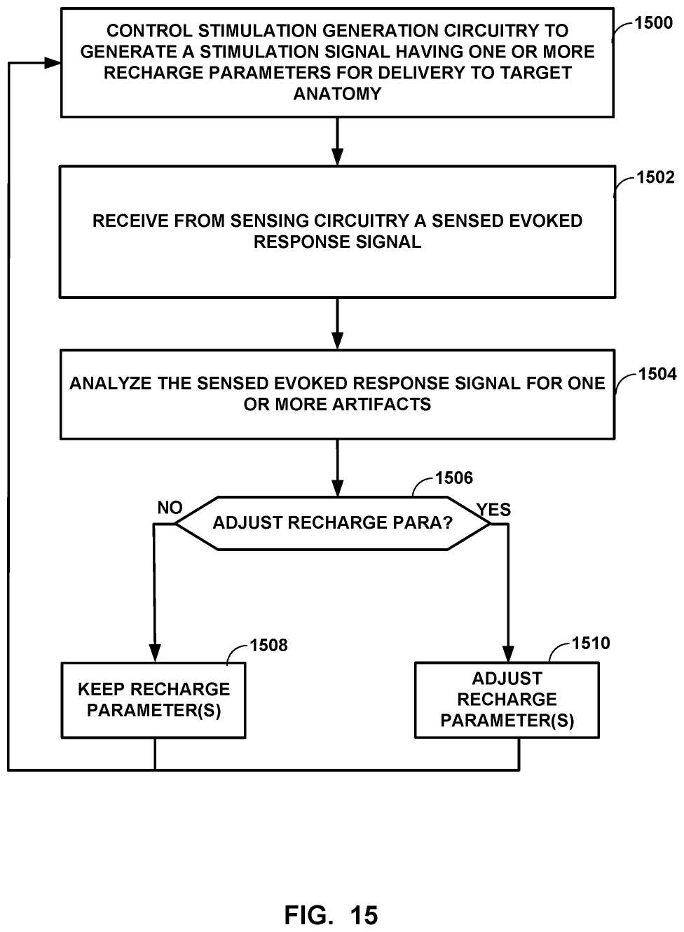

FIG. 15 is a flow diagram illustrating techniques to control artifact and electrode potential via a recharge phase. While the example of FIG. 15 is described with respect to IMD 200 of FIG. 2A, the techniques of FIG. 15 may be performed by any device capable of performing such techniques. Processing circuitry 210 may control stimulation generation circuitry 202 to generate a stimulation signal having one or more stimulation recharge parameters for delivery to target anatomy (1500). For example, processing circuitry 210 may control stimulation generation circuitry 202 to generate a stimulation signal having a first stimulation recharge parameter. Stimulation recharge parameters may include an active recharge parameter, a passive recharge parameter, or both an active recharge parameter and a passive recharge parameter. Active recharge parameters may include, but are not limited to, a duration of a recharge pulse, an amplitude of the recharge pulse, and/or a shape of the recharge pulse. Passive recharge parameters may include, but are not limited to, a duration of passive recharge.

Processing circuitry 210 may receive from sensing circuitry 206 a sensed evoked response signal (1502). For example, sensing circuitry 206 may sense an evoked response and send a sensed evoked response signal to processing circuitry 210. Processing circuitry 210 may analyze the sensed evoked response signal for one or more artifacts (1504). For example, processing circuitry 210 may analyze aspects or characteristics of any artifacts in the sensed evoked response signal. Processing circuitry may determine whether to adjust the stimulation recharge parameter(s) (1506). For example, processing circuitry 210 may determine a slope of an artifact, determine a total area under the curve of the artifact, determine a curvature of the artifact, determine a time constant of the artifact, determine a presence or absence of amplifier saturation to determine whether to adjust the stimulation recharge parameters. Processing circuitry 210 may compare any or all of such determinations against respective thresholds when determining whether to adjust the stimulation recharge parameter(s). In some examples, processing circuitry 210 may determine whether there is consistent noise, a relatively large degree of variability, a change over time, or an unsafe level of electrode potential, to determine whether to adjust the stimulation recharge parameter(s). In some examples, processing circuitry 210 may use sensing circuitry 206 to measure an impedance and determine whether to adjust the stimulation recharge parameter(s) based on the measured impedance.

If processing circuitry 210 determines not to adjust the stimulation recharge parameter(s) (the “No” path from box 1506), processing circuitry 210 may keep the current stimulation recharge parameter(s) (1508). If processing circuitry 210 determines to adjust the recharge parameter(s) (the “Yes” path from box 1506), processing circuitry 210 may adjust the current stimulation recharge parameter(s) (1510). For example, processing circuitry 210 may adjust, based on the one or more artifacts, a first stimulation recharge parameter to determine a second stimulation recharge parameter. Processing circuitry 210 may then control stimulation generation circuitry 202 to generate a second stimulation signal having the second stimulation recharge parameter for delivery to the target anatomy. For example, the second stimulation recharge parameter may result in a truncated (or paused) passive recharge phase, a relatively long period of time before another stimulation pulse, a different active recharge phase, or some combination thereof.

In any event, processing circuitry 210 may continue analyzing the sensed evoked response signal for one or more artifacts and adjusting parameters as needed as both box 1508 and box 1510 lead back to box 1500. Processing circuitry 210 may analyze the sensed evoked response signal, continuously, after each delivered stimulation pulse, periodically, in a time variable manner, or the like.

In some examples, the recharge phase may include a hybrid combination of both active and passive recharge. For example, IMD 110 may adjust both active and passive recharge parameters of a stimulation signal at the same time or one at a time.

In some examples, IMD 110 may employ recharge blanking. For example, IMD 110 may pause or truncate the recharge phase during sensing and restart the recharge phase once sensing is complete. In some examples, IMD 110 may employ recharge blanking with passive recharge, active recharge or hybrid active/passive recharge.

In some examples, each of the first stimulation signal and the second stimulation signal includes one of an active recharge phase, a passive recharge phase, or a hybrid active/passive recharge phase. In some examples, the first stimulation signal includes one of the active recharge phase, the passive recharge phase, or the hybrid active/passive recharge phase and wherein the second stimulation signal includes a different one of the active recharge phase, the passive recharge phase, or the hybrid active/passive recharge phase. For example, processing circuitry 210 may switch between different types of recharge (active recharge versus passive recharge or combination of both) or adjust other parameters of stimulating waveform, such as frequency, duration, pulse width, stimulating electrodes or time separation between stimulus and sensing window.

In some examples, the first stimulation signal includes one of the active recharge phase, the passive recharge phase, or the hybrid active/passive recharge phase, wherein the passive recharge phase includes a truncated or paused passive recharge phase and the hybrid active/passive recharge phase includes a hybrid active/truncated passive recharge phase or a hybrid active/paused passive recharge phase, wherein processing circuitry 210 is further configured to determine that delivery of stimulation is not time constrained, and wherein the second stimulation signal is a full passive recharge phase and wherein processing circuitry 210 adapts the first stimulation signal to be the full passive recharge phase based on the determination that the delivery of stimulation is not time constrained. For example, processing circuitry 210 may determine to switch to full passive recharge mode (e.g., without truncation or pausing) as an alternative to truncated or paused passive recharge if IMD 200 is not time constrained (e.g., the stimulation signal has a relatively slow stimulation frequency, such as a relatively long time between stimulation pulses).

In some examples, at least one of the first stimulation recharge parameter or the second stimulation recharge parameter includes a duration of passive recharge, a duration of a recharge pulse, an amplitude of the recharge pulse, or a shape of the recharge pulse. In some examples, processing circuitry 210 is further configured to recursively analyze the sensed evoked response signal for one or more artifacts, wherein a time period between consecutive recursive analyses varies. For example, processing circuitry 210 may, at certain times, monitor any artifacts more closely (e.g., the determining whether an artifact adjustment is needed is a lower threshold) or more frequently, while at other times, processing circuitry 210 may monitor the artifacts less closely (e.g., the determining whether an artifact adjustment is needed is a higher threshold) or less frequently, such as for an occasional “tune-up” or at a fixed value.

In some examples, processing circuitry 210 determines that a request to perform a recharge adjustment has been received by telemetry circuitry 208 to trigger analyzing the sensed evoked response signal for one or more artifacts and wherein the processing circuitry analyzes the sensed evoked response signal for the one or more artifacts based on the received request to perform the recharge adjustment. For example, recharge adjustment may be triggered on demand (e.g., by a clinician or patient 105 via external programmer 150).

In some examples, processing circuitry 210 determines to analyze the sensed evoked response signal in response to at least one of a change in stimulation parameters, a change in patient posture, or a change in patient activity level. For example, processing circuitry 210 may adjust stimulation recharge parameters when stimulation parameters change (such as during ECAP closed loop mode or when patient 105 adjusts stimulation parameters). In some examples, processing circuitry 210 may adjust stimulation recharge parameters when patient 105 posture and/or activity levels change.

In some examples, the one or more artifacts includes a plurality of artifacts, and processing circuitry 210 independently evaluates each of the plurality of artifacts. In some examples, processing circuitry 210 independently evaluates each of the plurality of artifacts using superposition of each of the plurality of artifacts by toggling on and off specific stimulus one at a time. For example, when multiple sources of artifacts are present (for example, if several stimulation waveforms are applied simultaneously or close in time on different pair of electrodes), processing circuitry 210 may independently evaluate each artifact. In some examples, processing circuitry 210 may independently adjust stimulation recharge parameters for stimulation signals from a plurality of different electrode combinations.

In some examples, analyzing the sensed evoked response signal for the one or more artifacts includes processing circuitry 210 determining at least one of a slope of an artifact, a total area under the curve of the artifact, a curvature of the artifact, a time constant of the artifact, or a presence or absence of amplifier saturation. Processing circuitry 210 may compare the at least one of the slope of an artifact, the total area under the curve of the artifact, the curvature of the artifact, the time constant of the artifact, or the presence or absence of amplifier saturation to a respective threshold, wherein adjusting the first stimulation recharge parameter to determine a second stimulation recharge parameter is based on the comparison.

In some examples, if the first stimulation signal includes an active recharge phase, processing circuitry 210 may, based on a battery charge level reaching a threshold, adjust the first stimulation recharge parameter to include a passive recharge phase, in order to extend the battery charge or battery life. For example, if the battery charge level is relatively low and the first stimulation signal includes an active recharge phase, processing circuitry 210 may adjust the first stimulation recharge parameter to determine a second stimulation recharge parameter for the second stimulation signal that does not include an active recharge phase.

The techniques of this disclosure may be particularly useful because a direct current (DC) connection on the electrodes is not necessary to assess the potentials of the electrodes. Such information may be obtained (or a proxy therefore may be obtained) from the artifact parameters (for example, slope) which may be sensed with amplifier(s) 233 of sensing circuitry 206 (both of FIG. 2). These techniques may be very useful, as an IMD, such as IMD 110 or IMD 200, may optimize or improve stimulation recharge parameters such that an operating point of an electrode after stimulus is closer to the electrode's open circuit potential (OCP) or stable point of operation.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various operations and functions described in this disclosure. For example, processing circuitry may conduct processing off-line and conduct automatic checks of patient ECAP signals and update programming from a remote location. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

This disclosure includes the following non-limiting examples.

Example 1. A device comprising: stimulation generation circuitry configured to generate a first stimulation signal, according to a set of parameters to be delivered to target anatomy of a patient via a stimulation electrode configuration from a plurality of electrodes, the set of parameters comprising a first stimulation recharge parameter; sensing circuitry configured to sense an evoked response signal responsive to the stimulation signal; processing circuitry communicatively coupled to the stimulation generation circuitry and the sensing circuitry, the processing circuitry being configured to: control the stimulation generation circuitry to generate the first stimulation signal having the first stimulation recharge parameter for delivery to the target anatomy; receive from the sensing circuitry the sensed evoked response signal; analyze the sensed evoked response signal for one or more artifacts; adjust, based on the one or more artifacts, the first stimulation recharge parameter to determine a second stimulation recharge parameter; and control the stimulation generation circuitry to generate a second stimulation signal having the second stimulation recharge parameter for delivery to the target anatomy.

Example 2. The device of example 1, wherein each of the first stimulation signal and the second stimulation signal comprises one of an active recharge phase, a passive recharge phase, or a hybrid active/passive recharge phase.

Example 3. The device of example 2, wherein the first stimulation signal comprises one of the active recharge phase, the passive recharge phase, or the hybrid active/passive recharge phase and wherein the second stimulation signal comprises a different one of the active recharge phase, the passive recharge phase, or the hybrid active/passive recharge phase.

Example 4. The device of example 3, wherein the first stimulation signal comprises one of the active recharge phase, the passive recharge phase, or the hybrid active/passive recharge phase, wherein the passive recharge phase comprises a truncated or paused passive recharge phase and the hybrid active/passive recharge phase comprises a hybrid active/truncated passive recharge phase or a hybrid/paused passive recharge phase, wherein the processing circuitry is further configured to determine that delivery of stimulation is not time constrained, and wherein the second stimulation signal is a full passive recharge phase and wherein the processing circuitry adapts the first stimulation signal to be the full passive recharge phase based on the determination that the delivery of stimulation is not time constrained.

Example 5. The device of any of examples 1-4, wherein at least one of the first stimulation recharge parameter or the second stimulation recharge parameter comprises a duration of passive recharge, a duration of a recharge pulse, an amplitude of the recharge pulse, or a shape of the recharge pulse.

Example 6. The device of any of examples 1-5, wherein the processing circuitry is further configured to recursively analyze the sensed evoked response signal for one or more artifacts, wherein a time period between consecutive recursive analyses varies.

Example 7. The device of any of examples 1-5, wherein the device further comprises telemetry circuitry and wherein the processing circuitry is further configured to determine that a request to perform a recharge adjustment has been received by the telemetry circuitry to trigger analyzing the sensed evoked response signal for one or more artifacts and wherein the processing circuitry analyzes the sensed evoked response signal for the one or more artifacts based on the received request to perform the recharge adjustment.

Example 8. The device of any of examples 1-5, wherein the processing circuitry is further configured to determine to analyze the sensed evoked response signal in response to at least one of a change in stimulation parameters, a change in patient posture, or a change in patient activity level.

Example 9. The device of any of examples 1-8, wherein the one or more artifacts comprises a plurality of artifacts, and wherein the processing circuitry is further configured to independently evaluate each of the plurality of artifacts.

Example 10. The device of example 9, wherein as part of independently evaluating each of the plurality of artifacts, the processing circuitry is configured to use superposition of each of the plurality of artifacts by toggling on and off specific stimulus one at a time.

Example 11. The device of any of examples 1-10, wherein as part of analyzing the sensed evoked response signal for the one or more artifacts, the processing circuitry if configured to: determine at least one of a slope of an artifact, a total area under the curve of the artifact, a curvature of the artifact, a time constant of the artifact, or a presence or absence of amplifier saturation; and compare the at least one of the slope of an artifact, the total area under the curve of the artifact, the curvature of the artifact, the time constant of the artifact, or the presence or absence of amplifier saturation to a respective threshold, wherein adjusting the first stimulation recharge parameter to determine a second stimulation recharge parameter is based on the comparison.

Example 12. A method comprising: controlling, by processing circuitry, stimulation generation circuitry to generate the first stimulation signal having a first stimulation recharge parameter for delivery to target anatomy of a patient; receiving, by the processing circuitry and from sensing circuitry, a sensed evoked response signal; analyzing, by the processing circuitry, the sensed evoked response signal for one or more artifacts; adjusting, by the processing circuitry and based on the one or more artifacts, the first stimulation recharge parameter to determine a second stimulation recharge parameter; and controlling, by the processing circuitry, the stimulation generation circuitry to generate a second stimulation signal having the second stimulation recharge parameter for delivery to the target anatomy.

Example 13. The method of example 12, wherein each of the first stimulation signal and the second stimulation signal comprises one of an active recharge phase, a passive recharge phase, or a hybrid active/passive recharge phase.

Example 14. The method of example 13, wherein the first stimulation signal comprises one of the active recharge phase, the passive recharge phase, or the hybrid active/passive recharge phase and wherein the second stimulation signal comprises a different one of the active recharge phase, the passive recharge phase, or the hybrid active/passive recharge phase.

Example 15. The method of example 14, wherein the first stimulation signal comprises one of the active recharge phase, the passive recharge phase, or the hybrid active/passive recharge phase, wherein the passive recharge phase comprises a truncated or paused passive recharge phase and the hybrid active/passive recharge phase comprises a hybrid active/truncated passive recharge phase or a hybrid active/paused passive recharge phase, the method further comprising determining, by the processing circuitry, that delivery of stimulation is not time constrained, and wherein the second stimulation signal is a full passive recharge phase and wherein adapting the first stimulation signal to be the full passive recharge phase is based on the determination that the delivery of stimulation is not time constrained.

Example 16. The method of any of examples 12-15, wherein at least one of the first stimulation recharge parameter or the second stimulation recharge parameter comprises a duration of passive recharge, a duration of a recharge pulse, an amplitude of the recharge pulse, or a shape of the recharge pulse.

Example 17. The method of any of examples 12-16, further comprising recursively analyzing the sensed evoked response signal for one or more artifacts, wherein a time period between consecutive recursive analyses varies.

Example 18. The method of any of examples 12-16, further comprising: determining, by the processing circuitry, that a request to perform a recharge adjustment has been received by telemetry circuitry prior to analyzing the sensed evoked response signal for one or more artifacts, and analyzing the sensed evoked response signal for the one or more artifacts is based on the received request to perform the recharge adjustment.

Example 19. The method of any of examples 12-16, further comprising determining, by the processing circuitry, to analyze the sensed evoked response signal in response to at least one of a change in stimulation parameters, a change in patient posture, or a change in patient activity level.

Example 20. The method of any of examples 12-19, wherein the one or more artifacts comprises a plurality of artifacts, and wherein the method further comprises independently evaluating, by the processing circuitry, each of the plurality of artifacts.

Example 21. The method of example 20, wherein independently evaluating each of the plurality of artifacts comprises using superposition of each of the plurality of artifacts by toggling on and off specific stimulus one at a time.

Example 22. The method of any of examples 12-21, wherein analyzing the sensed evoked response signal for the one or more artifacts comprises: determining, by the processing circuitry, at least one of a slope of an artifact, a total area under the curve of the artifact, a curvature of the artifact, a time constant of the artifact, or a presence or absence of amplifier saturation; and comparing, by the processing circuitry, the at least one of the slope of an artifact, the total area under the curve of the artifact, the curvature of the artifact, the time constant of the artifact, or the presence or absence of amplifier saturation to a respective threshold, wherein adjusting the first stimulation recharge parameter to determine a second stimulation recharge parameter is based on the comparison.

Example 23. A non-transitory computer-readable storage medium including instructions, which, when executed, cause processing circuitry to: control stimulation generation circuitry to generate a first stimulation signal having a first stimulation recharge parameter for delivery to target anatomy; receive from sensing circuitry a sensed evoked response signal; analyze the sensed evoked response signal for one or more artifacts; adjust, based on the one or more artifacts, the first stimulation recharge parameter to determine a second stimulation recharge parameter; and control the stimulation generation circuitry to generate a second stimulation signal having the second stimulation recharge parameter for delivery to the target anatomy.

Various examples have been described in the disclosure. These and other examples are within the scope of the following claims.

What is claimed is:

1. A device comprising:
stimulation generation circuitry configured to generate a first stimulation signal according to a set of parameters to be delivered to target anatomy of a patient via a stimulation electrode configuration from a plurality of electrodes, the set of parameters comprising a first stimulation recharge parameter;
sensing circuitry configured to sense an evoked response signal responsive to the first stimulation signal;
processing circuitry communicatively coupled to the stimulation generation circuitry and the sensing circuitry, the processing circuitry being configured to:
control the stimulation generation circuitry to generate the first stimulation signal having the first stimulation recharge parameter for delivery to the target anatomy;
receive from the sensing circuitry the sensed evoked response signal;
analyze the sensed evoked response signal for one or more artifacts;
adjust, based on the one or more artifacts, the first stimulation recharge parameter to determine a second stimulation recharge parameter; and
control the stimulation generation circuitry to generate a second stimulation signal having the second stimulation recharge parameter for delivery to the target anatomy.

2. The device of claim 1, wherein each of the first stimulation signal and the second stimulation signal comprises one of an active recharge phase, a passive recharge phase, or a hybrid active/passive recharge phase.

3. The device of claim 2, wherein the first stimulation signal comprises one of the active recharge phase, the passive recharge phase, or the hybrid active/passive recharge phase and wherein the second stimulation signal comprises a different one of the active recharge phase, the passive recharge phase, or the hybrid active/passive recharge phase.

4. The device of claim 3, wherein the first stimulation signal comprises one of the active recharge phase, the passive recharge phase, or the hybrid active/passive recharge phase, wherein the passive recharge phase comprises a truncated or paused passive recharge phase and the hybrid active/passive recharge phase comprises a hybrid active/truncated passive recharge phase or a hybrid active/paused passive recharge phase, wherein the processing circuitry is further configured to determine that delivery of stimulation is not time constrained, and wherein the second stimulation signal is a full passive recharge phase and wherein the processing circuitry adapts the first stimulation signal to be the full passive recharge phase based on the determination that the delivery of stimulation is not time constrained.

5. The device of claim 1, wherein at least one of the first stimulation recharge parameter or the second stimulation recharge parameter comprises a duration of passive recharge, a duration of a recharge pulse, an amplitude of the recharge pulse, or a shape of the recharge pulse.

6. The device of claim 1, wherein the processing circuitry is further configured to recursively analyze the sensed evoked response signal for one or more artifacts, wherein a time period between consecutive recursive analyses varies.

7. The device of claim 1, wherein the device further comprises telemetry circuitry and wherein the processing circuitry is further configured to determine that a request to perform a recharge adjustment has been received by the telemetry circuitry to trigger analyzing the sensed evoked response signal for one or more artifacts and wherein the processing circuitry analyzes the sensed evoked response signal for the one or more artifacts based on the received request to perform the recharge adjustment.

8. The device of claim 1, wherein the processing circuitry is further configured to determine to analyze the sensed evoked response signal in response to at least one of a change in stimulation parameters, a change in patient posture, or a change in patient activity level.

9. The device of claim 1, wherein the one or more artifacts comprises a plurality of artifacts, and wherein the processing circuitry is further configured to independently evaluate each of the plurality of artifacts.

10. The device of claim 9, wherein as part of independently evaluating each of the plurality of artifacts, the processing circuitry is configured to use superposition of each of the plurality of artifacts by toggling on and off specific stimulus one at a time.

11. The device of claim 1, wherein as part of analyzing the sensed evoked response signal for the one or more artifacts, the processing circuitry if configured to:
determine at least one of a slope of an artifact, a total area under a curve of the artifact, a curvature of the artifact, a time constant of the artifact, or a presence or absence of amplifier saturation; and
compare the at least one of the slope of an artifact, the total area under the curve of the artifact, the curvature of the artifact, the time constant of the artifact, or the presence or absence of amplifier saturation to a respective threshold, wherein adjusting the first stimulation recharge parameter to determine a second stimulation recharge parameter is based on the comparison.

12. A method comprising:
controlling, by processing circuitry, stimulation generation circuitry to generate a first stimulation signal having a first stimulation recharge parameter for delivery to target anatomy of a patient;
receiving, by the processing circuitry and from sensing circuitry, a sensed evoked response signal;
analyzing, by the processing circuitry, the sensed evoked response signal for one or more artifacts;
adjusting, by the processing circuitry and based on the one or more artifacts, the first stimulation recharge parameter to determine a second stimulation recharge parameter; and
controlling, by the processing circuitry, the stimulation generation circuitry to generate a second stimulation signal having the second stimulation recharge parameter for delivery to the target anatomy.

13. The method of claim 12, wherein each of the first stimulation signal and the second stimulation signal comprises one of an active recharge phase, a passive recharge phase, or a hybrid active/passive recharge phase.

14. The method of claim 13, wherein the first stimulation signal comprises one of the active recharge phase, the passive recharge phase, or the hybrid active/passive recharge phase and wherein the second stimulation signal comprises a different one of the active recharge phase, the passive recharge phase, or the hybrid active/passive recharge phase.

15. The method of claim 14, wherein the first stimulation signal comprises one of the active recharge phase, the passive recharge phase, or the hybrid active/passive recharge phase, wherein the passive recharge phase comprises a truncated or paused passive recharge phase and the hybrid active/passive recharge phase comprises a hybrid active/truncated passive recharge phase or a hybrid active/paused recharge phase, the method further comprising determining, by the processing circuitry, that delivery of stimulation is not time constrained, and wherein the second stimulation signal is a full passive recharge phase and wherein adapting the first stimulation signal to be the full passive recharge phase is based on the determination that the delivery of stimulation is not time constrained.

16. The method of claim 12, wherein at least one of the first stimulation recharge parameter or the second stimulation recharge parameter comprises a duration of passive recharge, a duration of a recharge pulse, an amplitude of the recharge pulse, or a shape of the recharge pulse.

17. The method of claim 12, further comprising recursively analyzing the sensed evoked response signal for one or more artifacts, wherein a time period between consecutive recursive analyses varies.

18. The method of claim 12, further comprising:
determining, by the processing circuitry, that a request to perform a recharge adjustment has been received by telemetry circuitry prior to analyzing the sensed evoked response signal for one or more artifacts, and analyzing the sensed evoked response signal for the one or more artifacts is based on the received request to perform the recharge adjustment.

19. The method of claim 12, further comprising determining, by the processing circuitry, to analyze the sensed evoked response signal in response to at least one of a change in stimulation parameters, a change in patient posture, or a change in patient activity level.

20. The method of claim 12, wherein the one or more artifacts comprises a plurality of artifacts, and wherein the method further comprises independently evaluating, by the processing circuitry, each of the plurality of artifacts.

21. The method of claim 20, wherein independently evaluating each of the plurality of artifacts comprises using superposition of each of the plurality of artifacts by toggling on and off specific stimulus one at a time.

22. The method of claim 12, wherein analyzing the sensed evoked response signal for the one or more artifacts comprises:
determining, by the processing circuitry, at least one of a slope of an artifact, a total area under a curve of the artifact, a curvature of the artifact, a time constant of the artifact, or a presence or absence of amplifier saturation; and
comparing, by the processing circuitry, the at least one of the slope of an artifact, the total area under the curve of the artifact, the curvature of the artifact, the time constant of the artifact, or the presence or absence of amplifier saturation to a respective threshold, wherein adjusting the first stimulation recharge parameter to determine a second stimulation recharge parameter is based on the comparison.

23. A non-transitory computer-readable storage medium including instructions, which, when executed, cause processing circuitry to:
control stimulation generation circuitry to generate a first stimulation signal having a first stimulation recharge parameter for delivery to target anatomy;
receive from sensing circuitry a sensed evoked response signal;
analyze the sensed evoked response signal for one or more artifacts;
adjust, based on the one or more artifacts, the first stimulation recharge parameter to determine a second stimulation recharge parameter; and
control the stimulation generation circuitry to generate a second stimulation signal having the second stimulation recharge parameter for delivery to the target anatomy.
